(19) United States
(12) Patent Application Publication
(10) Pub. No.: US 2024/0363201 A1
(43) Pub. Date: Oct. 31, 2024

(54) SYSTEM AND METHOD FOR SIMULATION OF MARINE POLLUTION DISPERSION USING NUMERICAL TRACER TECHNIQUE

(71) Applicant: KOREA INSTITUTE OF OCEAN SCIENCE & TECHNOLOGY, Busan (KR)

(72) Inventors: Moonjin LEE, Daejeon (KR); Tae Sung KIM, Daejeon (KR); Yong Myung KIM, Daejeon (KR)

(73) Assignee: KOREA INSTITUTE OF OCEAN SCIENCE & TECHNOLOGY, Busan (KR)

(21) Appl. No.: 18/604,072

(22) Filed: Mar. 13, 2024

(30) Foreign Application Priority Data
Apr. 28, 2023 (KR) 10-2023-0056085

(51) Int. Cl. G16C 20/30; G16C 20/70

(52) U.S. Cl. CPC G16C 20/30; G16C 20/70

(57) ABSTRACT

Proposed is a system and method for simulations of marine pollution dispersion using a numerical tracer technique, wherein modeling is faster than conventional diffusion modeling methods by reducing computational volume and load and thereby a simulation of marine pollution dispersion with respect to the long-term continuous discharge may be performed through the numerical tracer technique by configuring to simulate the marine dispersion of suspended sediments (SS) and COD using the Quick Dispersion (Q-DISP) model, which is a diffusion model using the Monte Carlo method, in order to solve the problems of conventional marine pollution dispersion modeling systems and methods using numerical tracer techniques which have the disadvantage of requiring a high-performance computer and a large amount of memory due to the increased computation.

### CROSS REFERENCE TO RELATED APPLICATION

The present application claims priority to Korean Patent Application No. 10-2023-0056085, filed on Apr. 28, 2023, the entire contents of which are incorporated herein for all purposes by this reference.

### BACKGROUND

### Technical Field

The present disclosure relates to a system and method for simulating the dispersion of marine pollutants and, more specifically, to a system and method for simulation of marine pollution dispersion using a numerical tracer technique, which is configured to simulate marine pollution dispersion using a numerical tracer technique that discharges a numerical tracer instead of a physical tracer at the location of a pollution source in order to apply the intensity of the pollution source in the diffusion model in accordance with reality, and reconstructs the dispersion of pollutants by tracing the location of each particle, thereby solving the problems of conventional marine pollution dispersion modeling systems and methods using a finite difference method due to a numerical diffusion problem of an advection term and the practical difficulties in constructing a fine grid that specifies the appropriate concentration of a pollution source.

In addition, the present disclosure relates to a system and method for simulations of marine pollution dispersion using a numerical tracer technique, wherein modeling is faster than conventional diffusion modeling methods by reducing computational volume and load and thereby the simulation of marine pollution dispersion is performed with respect to the long-term continuous discharge through the numerical tracer technique that is configured to simulate the marine dispersion of suspended sediments (SS) and COD using the Quick Dispersion (Q-DISP) model, which is a diffusion model using the Monte Carlo method, in order to solve the problems of conventional marine pollution dispersion modeling systems and methods using numerical tracer techniques, where accurate simulation is possible using numerical tracers instead of physical tracers, but there is an disadvantage of requiring a high-performance computer and a large amount of memory due to increased computation, which makes it difficult to apply to a pollution source that continuously discharges pollutants over a long period of time.

### DESCRIPTION OF THE RELATED ART

Recently, as environmental pollution and various environmental problems become more serious day by day, interest and demand for eco-friendliness are increasing, and technology developments to simulate and trace the inflow and dispersion of various marine pollutants are being actively carried out in order to effectively prevent the inflow of marine pollutants into the sea and efficiently remove the marine pollutants that have already entered the sea.

That is, various devices and methods have been proposed to trace the discharge and dispersion of marine pollutants, such as, for example, “Automatic Generation-Display System of Marine Pollution Information and Method thereof” in Korean Patent No. 10-2469002 and “Apparatus and Method for Tracking Location Using control Platform of Small Buoy for Simulating marine Pollutants” in Korean Patent No. 10-2334171, but the contents of conventional technology as described above have the following limitations.

More specifically, in general, pollutants flowing into the ocean are advectively moved along the flow of tidal currents and ocean currents, and an eddy dispersion occurs simultaneously due to turbulent flows of seawater.

Accordingly, in order to model the dispersion of pollutants in seawater, first the modeling of seawater flow is essentially required and such modeling data of seawater flows are utilized as input data for pollutant dispersion models.

In addition, pollutants flowing into the ocean are divided into conservative and non-conservative pollutants, that is, pollutants that are decomposed by chemical or biological action are non-conservative pollutants, and suspended sediments are also treated as non-conservative pollutants because suspended sediments gravitate to the bottom over time, so that the degree of the decomposition or reduction of pollutants should be appropriately reflected in order to model the dispersion of such non-conservative pollutants.

That is, when there is a continuous source of pollution, the concentration of pollutants initially increases over time, but after sufficient time has passed, an equilibrium state where the pollutant concentration does not increase will be reached when there is a balance between the amount of pollutant loads flowing into the ocean and the amount of pollutants escaping from the ocean through the decomposition or sedimentation.

Therefore, in pollutant dispersion modeling, the concentration distribution in the equilibrium state should be reproduced, but this requires modeling for an actual time that is more than three times the half-life of the pollutant decomposition.

Moreover, generally, numerical analysis techniques such as a finite difference diffusion modeling that performs a modeling through a diffusion equation in a Eulerian coordinate system or a Lagrangian coordinate system are mainly used to conventionally model the dispersion of marine pollutants, but these techniques using the finite difference method have a numerical diffusion problem of the advection term and the practical difficulties in constructing fine grids that are capable of specifying the appropriate concentration of a pollution source.

In order to solve the above-mentioned problems and apply the intensity of a pollution source in a diffusion model in accordance with reality, a numerical tracer technique has been proposed to discharge a numerical tracer instead of the conventional physical tracer from the location of the pollution source and reconstruct the dispersion of pollutants by tracing the location of each particle.

However, such a conventional numerical tracer technique has a disadvantage of requiring a high-performance computer and a large amount of memory due to the increased amount of computation and accordingly, has a limitation that makes it difficult to apply, for example, to a pollution source that discharges pollutants continuously over a long period of time, due to computer capacity and memory problems.

Therefore, in order to solve the limitations of conventional systems and methods for simulations of marine pollution dispersion, it is desirable to present a system and method for simulations of marine pollution dispersion using a new configuration of a numerical tracer technique that performs simulations by applying the numerical tracer technique even to the dispersion of marine pollution that is continuously discharged for a long time by reducing the amount of computation and load, but no device or technique has been proposed to satisfy all such needs.

### DOCUMENTS OF RELATED ART

(Patent Document 1) Korean Patent No. 10-2469002 (Nov. 18, 2022)
(Patent Document 2) Korean Patent No. 10-2334171 (Dec. 2, 2021)

### SUMMARY

In order to solve the problems of conventional works as described above, the objective of the present disclosure is to propose a system and method using a numerical tracer technique, which simulates marine pollution dispersion using a numerical tracer technique that discharges a numerical tracer instead of a physical tracer at a location of a pollution source in order to apply the intensity of the pollution source in a diffusion model in accordance with reality, and reconstructs the dispersion of pollutants by tracing the location of each particle, thereby solving the problems of conventional systems and methods for modeling marine pollution dispersion using a finite difference method, which have the numerical diffusion problem of the advection term and the practical difficulties in constructing fine grids that are capable of specifying the appropriate concentration of a pollution source.

In addition, another objective of the present disclosure is to propose a system and method for simulations of marine pollution dispersion using a numerical tracer technique, wherein modeling is faster than conventional diffusion modeling methods by reducing computational volume and load and thereby simulations of marine pollution dispersion with respect to the long-term continuous discharge may be performed through the numerical tracer technique that is configured to simulate the marine dispersion of suspended sediments (SS) and COD using the Quick Dispersion (Q-DISP) model, which is a diffusion model using the Monte Carlo method, in order to solve the problems of conventional systems and methods for modeling marine pollution dispersion using numerical tracer techniques, in which more accurate simulation is possible by using numerical tracers instead of physical tracers but there is an disadvantage of requiring a high-performance computer and a large amount of memory due to the increased computation, which makes it more difficult to apply to a pollution source that continuously discharges pollutants over a long period of time.

In order to achieve the above objectives, a system for simulations of marine pollution dispersion using the numerical tracer technique according to the present disclosure may include a data collection unit that performs a process of collecting various data predetermined in order to perform simulations on marine pollution dispersion, a simulation processing unit that performs simulations of marine pollution dispersion according to a predetermined setting using the numerical tracer technique on the basis of various data collected through the data collection unit, and an output unit that performs a process of outputting various data, each processing procedure, and the processing results that are collected and processed through the data collection unit and the simulation processing unit according to the predetermined settings.

Herein, the system may further include a communication unit that performs a process of communicating with an external device including a server or a user terminal through at least one of wired or wireless communications in order to transmit and receive various data and a controller that performs a process of controlling the overall operations of the system.

In addition, the data collection unit may be configured to receive through a separate input means directly or externally from the outside the basic data such as grids, depths, tides and current information for implementing a hydrodynamic model of a region to be simulated, the data on environmental conditions, or the data on pre-implemented hydrodynamic models about the corresponding region.

Moreover, the simulation processing unit may include a step of pollutant dispersion modeling in which an advection and dispersion of pollutants are modeled by calculating the displacement of pollutants per unit time using the Monte Carlo method, a step of eddy diffusion coefficient calculation in which an eddy diffusion coefficient involved in turbulent flows is estimated by calculating a neighbor diffusion coefficient, a step of pollutant sedimentation and decomposition modeling in which the sedimentation of suspended sediments (SS) and the decomposition of COD are modeled using a relational equation for the number of particles in seawater, and a step of pollutant concentration calculation in which the pollutant concentration in seawater is quantitatively calculated using the number of numerical tracers in each grid and the mass of pollutants.

Herein, using the following mathematical equations, the step of pollutant dispersion modeling may perform a process of calculating the displacement (δx, δy) during the time δt by modeling turbulent flows using Monte Carlo method when the particles at the location (xo, yo) at the time t are moved by the mean flows (tidal currents and permanent currents) and turbulent vortices, and then locate at a new position (xo+δx, yo+δy) after δt hours.

$$δ x = ( U + μ {U}_{b} ) δ t$$

$$δ y = ( V + {vV}_{b} ) δ t$$

$${U}_{b} = {( 6 Dx / δ t )}^{1 / 2}$$

$${V}_{b} = {( 6 Dy / 6 t )}^{1 / 2}$$

where U and V are the velocity of mean flows calculated by the hydrodynamic model, y and v are respectively random numbers uniformly distributed in the range of −1 and 1, U_{b} and V_{b} are turbulent characteristic velocities, and Dx and Dy are respectively eddy diffusion coefficients in the x and y directions.

Furthermore, using the following mathematical equation, the step of eddy diffusion coefficient calculation may perform a process of calculating the neighbor diffusion coefficient D(σ) according to the variance σ² of the distance between the buoys during the time t.

$$D ( σ ) = {σ}^{2} / 2 t$$

In addition, using the following mathematical equation, the step of pollutant sedimentation and decomposition modeling may perform a process of calculating the number of suspended sediment particles (N) per water column of a unit area by the sedimentation of suspended sediments present in the seawater and the number of suspended sediment particles N(t) at the time t,

$$\frac{d N}{d t} = - α N$$

$$N ( t ) = {N}_{0} exp ( - α t ) = {N}_{0} exp ( \frac{- t}{{t}_{0}} )$$

where α is the attenuation coefficient, N₀ is the initial number of particles, and t₀(t₀=1/α) means an exponential half-life (e-folding time) or residence time, respectively, for the number of particles to be decreased by a factor of e⁻¹(=0.368), and modeling both whether to be accumulated by the sedimentation of suspended sediment particles and the concentration reduction according to the decomposition of COD in seawater on the basis of the value of the uniformly distributed random number using the relationship between the number of particles N(n+1) in the time (t+δt) and the number of particles N(n) in the time t, which is expressed by the following equation.

$$N ( n + 1 ) = N ( n ) ( 1 - α δ t )$$

Moreover, the step of pollutant sedimentation and decomposition modeling may be configured to model the behavior of suspended sediment particles by treating the corresponding particles as being accumulated when the uniformly distributed random number taken arbitrarily between 0 and 1 is within the interval between 0 and αβt, and by treating the particles as continuously floating when the uniformly distributed random number is within the interval between αβt and 1.

Furthermore, the step of pollutant concentration calculation may perform a process of modeling the discharge of Q kg of pollutants per unit time as the discharge of m particles per unit time, wherein one particle represents the amount of pollutants in Q/m kg, and calculating the concentration (C) of pollutants in the seawater from the number of numerical tracers present in each grid using the following mathematical equation on the basis of the fact that the mass of pollutants present in a water column is nQ/m kg when there are n particles in the water column where the width, length, and depth are respectively δx, δy, and δz.

$$c = \frac{n Q}{m δ x δ y δ z}$$

Moreover, the simulation processing unit may be configured to perform simulations for actual time according to predetermined settings, taking into consideration the time to reach an equilibrium state in which the dispersion concentration of pollutants in seawater no longer increases.

In addition, the simulation processing unit may be configured to model the pollutant dispersion not by continuously discharging a numerical tracer from a pollution source but by estimating equivalently the dispersion state of later continuously discharged particles over time on the basis of the time-specific dispersion state of the initially discharged particles while simulating the dispersion according to continuous discharges of pollutants when the flow field is steady currents or periodically reversing currents, thereby reducing the amount of computational work and execution time required for modeling by tracing only the particles discharged during one tidal cycle using the periodicity of the tide and then by equivalently modeling the subsequent tidal cycle.

Moreover, the simulation processing unit may perform a process of modeling the pollutant dispersion by setting a buffer outside the hydrodynamic model area according to predetermined settings in order to consider the possibility that particles escaping from the predetermined hydrodynamic model area may reenter the corresponding model area again.

Furthermore, the simulation processing unit may perform a process of simultaneously modeling each pollutant particle by setting a different residence time for each pollutant particle according to a composition ratio per size of each pollutant particle and specifying a lifetime of each particle in advance when multiple pollutants having different residence times are mixed.

In addition, the output unit may perform a process of visually displaying various data received through the data collection unit and various information including the processing procedure and the results of the simulation processing unit through a monitor or display.

Moreover, the output unit may perform a process of visually displaying various information through a monitor or display, and at the same time, audibly transmitting various information through an audio output means including a speaker.

Moreover, the controller may perform a process of transmitting various data received through the data collection unit and various information including the processing procedure and the analysis results of the simulation processing unit to an external device including a server or a user terminal according to predetermined settings through the communication unit.

In addition, according to the present disclosure, a method for simulations of marine pollution dispersion using a numerical tracer technique may include a step of system construction that performs a process of constructing a simulation system where the simulation of marine pollution dispersion is performed using a numerical tracer technique and a step of simulation execution that performs the simulation of the marine pollution dispersion using the simulation system, wherein the simulation system is configured to use the marine pollution dispersion simulation system using the numerical tracer technique.

Moreover, a marine pollution dispersion simulation service supply system according to the present disclosure may include a marine pollution dispersion simulation execution unit that performs a process of performing simulations after receiving various information for simulations of marine pollution dispersion, and building a database related to simulations of marine pollution dispersion for each region by collecting various data including information received for simulation and simulation results, a user terminal for each user to input information related to simulations of marine pollution dispersion and request and receive a desired service, and a service server that performs a process of receiving various data including simulation results from the simulation execution unit and providing the corresponding services according to information received from each of the user terminal and the user's request while being interlocked with the simulation execution unit and the user terminal, wherein the marine pollution dispersion simulation execution unit is configured to use the system for simulations of marine pollution dispersion using the numerical tracer technique.

Herein, the user terminal may include at least one of a personal portable information communication terminal including a smartphone or a tablet PC, and an information processing device including a PC or a laptop.

According to the present disclosure as described above, proposed may be the system and method for simulations of marine pollution dispersion using a numerical tracer technique that simulates the marine dispersion of suspended sediments (SS), COD and the like using a Quick Dispersion (Q-DISP) model, which is a diffusion model using the Monte Carlo method, thereby reducing the amount of computation and load and making modeling faster than conventional diffusion modeling methods, accordingly resulting in enabling simulations using the numerical tracer technique with respect to the dispersion of marine pollution that is persistently discharged for a long time.

In addition, according to the present disclosure, the problems of conventional systems and methods for modeling marine pollution dispersion using numerical tracer techniques, in which more accurate simulation is possible by using numerical tracers instead of physical tracers but there is an disadvantage of requiring the high-performance computer and a large amount of memory due to the increased computation, which makes it more difficult to be applied to the pollution source that continuously discharges pollutants over a long period of time may be solved by providing the system and method for simulations of marine pollution dispersion using the numerical tracer technique, which is configured to enable simulations using the numerical tracer technique even for long-term continuous discharged pollutants by reducing the amount of calculation and load as described above.

Moreover, according to the present disclosure, the problems of conventional systems and methods for simulations of marine pollution dispersion modeling using the finite difference method, which have the limitations such as a numerical diffusion problem of advection terms and the practical difficulty in constructing fine grids that are capable of specifying the concentration of an appropriate pollution source may be solved by providing the system and method for simulations of marine pollution dispersion using the numerical tracer technique configured to enable simulation even for long-term continuously discharged pollutants using the numerical tracer technique by reducing the amount of computation and load, thereby enabling more accurate simulation compared to conventional methods.

### BRIEF DESCRIPTION OF THE DRAWINGS

FIG. 1 is a conceptual diagram schematically showing the overall process of hydrodynamic modeling and pollutant dispersion modeling required for marine pollutant dispersion modeling.

FIG. 2 is a table showing a sedimentation velocity according to a particle size of suspended sediments with a specific gravity of 2.65 in seawater with a water temperature of 20° C.

FIG. 3 is a block diagram schematically showing an overall configuration of a system for simulations of marine pollution dispersion using a numerical tracer technique according to an exemplary embodiment of the present disclosure.

FIG. 4 is a flowchart schematically showing an overall processing procedure performed in a simulation processing unit of a system for simulations of marine pollution dispersion using a numerical tracer technique according to an exemplary embodiment of the present disclosure shown in FIG. 3.

FIG. 5 is a block diagram schematically showing an overall configuration of a marine pollution dispersion simulation service supply system configured to provide a marine pollution dispersion simulation and various types of information related thereto using a system and method for simulations of marine pollution dispersion using a numerical tracer technique according to an exemplary embodiment of the present disclosure.

### DETAILED DESCRIPTION OF THE DISCLOSURE

Hereinafter, a detailed exemplary embodiment of a system and method for simulations of marine pollution dispersion using a numerical tracer technique according to the present disclosure will be described with reference to the accompanying drawings.

Here, it should be noted that the contents described below are only one exemplary embodiment for implementing the present disclosure, and the present disclosure is not limited to the contents of the exemplary embodiment described below.

In addition, it should be noted that in the description of an exemplary embodiment of the present disclosure below, the detailed description has been omitted to simplify the description of the parts that are the same as or similar to the contents of the related works, or that may be easily understood and implemented at the level of those skilled in the art.

Subsequently, detailed contents of a system and method for simulations of marine pollution dispersion using a numerical tracer technique according to the present disclosure will be described with reference to the drawings.

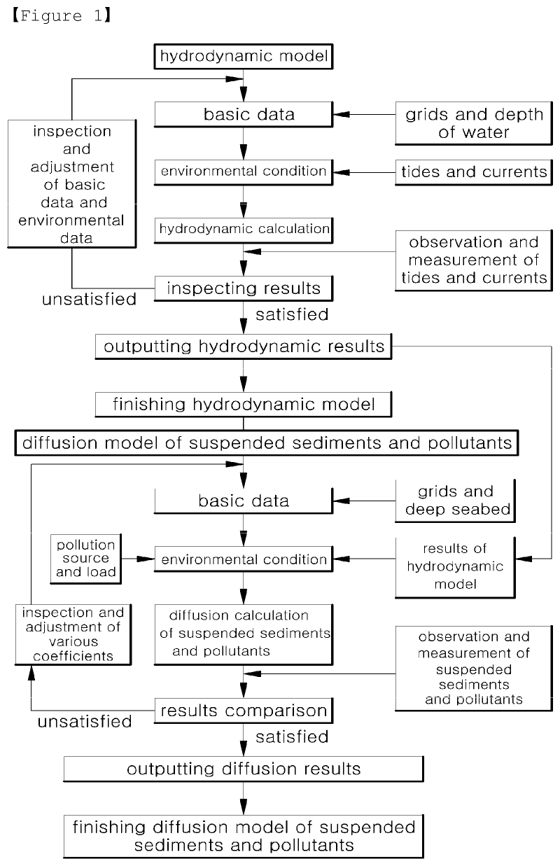

In more detail, first, referring to FIG. 1, FIG. 1 is a conceptual diagram schematically showing the overall processing procedure of hydrodynamic modeling and pollutant dispersion modeling required for marine pollutant dispersion modeling.

As shown in FIG. 1, pollutants flowing into the ocean may be advectively moved along the flow of tidal currents and ocean currents, and simultaneously eddy dispersion may occur due to turbulent flows of seawater, and accordingly, the modeling of seawater flow may be essentially required first and the data of hydrodynamic modeling may be utilized as input data for pollutant dispersion models in order to model the pollutant dispersion in seawater.

In addition, pollutants flowing into the ocean may be divided into conservative and non-conservative pollutants, that is, pollutants that are decomposed by chemical or biological action may be non-conservative pollutants, and suspended sediments may be also treated as non-conservative pollutants because suspended sediments gravitate to the bottom over time, so that the degree of decomposition or reduction of pollutants should be appropriately reflected in order to model the dispersion of such non-conservative pollutants.

When there is a continuous source of pollution, the concentration of pollutants may initially increase over time, but after sufficient time has passed, an equilibrium state where the pollutant concentration does not increase will be reached when there is a balance between the amount of pollutant loads flowing into the ocean and the amount of pollutants escaping from the ocean through the decomposition or sedimentation, and accordingly, the concentration distribution in the equilibrium state should be reproduced in pollutant dispersion modeling, and for this, the modeling for an actual time that is more than three times the half-life of the pollutant decomposition may be required.

More specifically, the basic equations of hydrodynamic modeling may be the two-dimensional hydrodynamic equation and the continuity equation, which may be represented mathematically as [Equation 1] below.

$$\frac{\partial U}{\partial t} + U \frac{\partial U}{\partial x} + V \frac{\partial U}{\partial y} + g \frac{\partial ζ}{\partial x} - f V + \frac{{k [ {U}^{2} + {V}^{2} ]}^{1 / 2} U}{H + ζ} = 0 \qquad [ Equation 1 ]$$

$$\frac{\partial V}{\partial t} + U \frac{\partial V}{\partial x} + V \frac{\partial V}{\partial y} + g \frac{\partial ζ}{\partial y} + f U + \frac{{k [ {U}^{2} + {V}^{2} ]}^{1 / 2} V}{H + ζ} = 0$$

$$\frac{\partial U}{\partial t} + \frac{\partial }{\partial x} [ ( H + ζ ) U ] + \frac{\partial }{\partial y} [ ( H + ζ ) V ] = 0$$

where t is the time, x and y are the distances on orthogonal coordinates, U and V are vertically averaged flow velocities in the x-axis and y-axis directions, g is gravitational acceleration, ζ is the sea level displacement from the mean sea level, H is the depth of water from the average sea level, f is the Coriolis parameter (f=2Ω sin ϕ), Ω is the Earth's rotational angular velocity (Ω=2π/86400 sec⁻¹), ϕ is the reference latitude, and k is the seabed friction coefficient (k=0.003).

In addition, conventionally, the finite difference or finite element method may be widely used to numerically reproduce the solution for the governing equations of seawater flow on a computer, and the finite element method, which represents the model of sea area using triangular meshes or “deformed” square meshes, may have an advantage of modeling and representing complex coastlines in a way that is almost similar to the real one, but have a disadvantage of being computationally intensive, requiring high-performance computers and consuming large amounts of memory, as the values of the physical variables at all nodal points connecting each finite element should be solved “simultaneously” using matrix expressions.

On the other hand, the finite difference method may have an advantage of being able to model even with a relatively small computer capacity, although it is difficult to accurately reflect the curved surface of the coastline due to the nature of the grid composed of right-angled squares.

In addition, techniques for finding a solution to a finite difference model may include an explicit scheme that calculates the value of each grid point in turn, and an implicit scheme that calculates the values of all grids simultaneously, and the explicit scheme may be advantageous in terms of computer capacity, but there may be a limitation of having to satisfy the stability condition that the time interval of the computation should be shorter than the time for the shallow-water long waves to propagate through adjacent grids.

On the other hand, the implicit scheme may not be limited by these stability conditions, but there may be a disadvantage in that the required computer performance and capacity and the time required for the computation may increase since the computations are performed using matrix expressions.

Therefore, in the present disclosure as described later, the finite difference method of the implicit scheme may be used for model calculation, an advective angled derivative scheme may be introduced to process advection terms, and the “double sweep scheme” may be applied in the calculation of the deflection force, seabed friction term, and the like to take advantage of the characteristics of the central divided difference and to offset and absorb numerical errors that occur in the process of calculating the numerical values of each stage.

Moreover, the sea-level change of the open boundary may be specified as a boundary condition in the hydrodynamic model, the boundary condition that the vertical constituent of the flow velocity is zero may be applied at the land boundary, the flow velocity according to the flow rate of the river water may be specified at the place where the river water flows in, and the boundary condition of the open sea may be input with the see-level displacement of each tidal constituent in the form of [Equation 2] below by referring to the tidal observation results.

$$ζ ( t ) = {ζ}_{1} cos ( ω t - {θ}_{1} ) \qquad [ Equation 2 ]$$

where ζ₁ is the amplitude of sea-level displacement, ω is the angular velocity (ω=28.9841042°/hr for M2 tidal constituent), and θ₁ is the phase of the sea-level displacement.

In addition, when only semi-diurnal tides are considered, the boundary condition according to [Equation 2] above may be sufficient, but when multiple tidal constituents are considered, the boundary condition of the open sea may be configured to apply the sea-level displacement by the synthesis of each tidal constituent instead of [Equation 2] described above.

Moreover, appropriate processing of the intertidal zone (tidal flat) may be very important in the hydrodynamic modeling of coastal shallow waters where the intertidal zone is widely developed, and for this purpose, for example, the processing technique presented by Flather and Heaps (1975) may be applied.

More specifically, the processing of the intertidal zone may be configured not to calculate the continuity equation when the corresponding calculation grid is exposed but to calculate the flow velocities (U, V) when being above a certain depth (e.g., 20 cm) by examining whether a grid is exposed after calculating the continuity equation.

At this time, the mass and quantity of motion may be not partially preserved in this process, but the benefits gained from processing the intertidal zone may be much greater than the losses, and when the intertidal zone is not inspected for dryness such that the intertidal zone may be regarded as land or regarded as sea, sufficient water depth values should be given to ensure that the intertidal zone does not expose the seafloor.

Furthermore, the ocean's tidal range may occur as a spring tide on the fifteenth day of the lunar month and on the last day of the lunar month, and as a neap tide when the moon is in the first or third quarter as the sun, earth and moon form a right angle, and this phenomenon may be attributed to the strengthening and attenuation of tidal movements according to the synthesis of the main lunar semi-diurnal tide (M2 tide) and the main solar semi-diurnal tide (S2 tide).

When considering the tide of the diurnal tide constituent, the phenomenon of daily tidal inequality may become more complex, and the technical method for reproducing the ocean tidal phenomenon according to the moon's phase may be as follows.

More specifically, as mentioned above, the method for applying the variations of tidal currents according to the moon's phase may be first to apply a boundary value by the synthesis of multiple tidal constituents, which is the most common method where the synthesis of multiple tidal constituents at the open sea boundary is input as a boundary condition and the tides inside the boundary area are modeled and simulated, but when modeling actual tides using this method, there may be a problem of requiring a lot of computer computation time since it is necessary to model the actual time corresponding to the least common multiple of the cycle of the considered various tidal constituents.

Next, there may be a method of synthesizing the results of each tidal constituent modeling, which reconstructs actual tides by separately calculating the results of tidal modeling for each tidal constituent and by synthesizing the results of these tidal constituents, and, in particular, storing and synthesizing the tidal harmonic constant values of tidal height and flow velocity corresponding to each tidal constituent may greatly reduce computer's memories and the computation time in approximately reproducing the tidal state at a specific time.

While these methods may be effectively used to reproduce tides in open waters where nonlinear actions may be almost ignored, careful consideration may be required in shallow water areas where nonlinear actions may not be ignored since these methods do not adequately reflect the influence of the nonlinear interaction of tidal currents of each tidal constituent.

Additionally, there may be a method of considering the tidal amplitude according to the moon's phase, the method which models the tides for the semi-diurnal tide in a sea area where the semi-diurnal cycle is dominant and then considers the degree to which the strength of these tides changes sinusoidally according to the moon's phase, and the results of this method may be slightly different from actual tides, but may be effectively used in the case of pollutant dispersion modeling in a general period that does not take into account a specific date in a sea area having a continuous pollution source.

Continuing to explain the modeling of the permanent current constituent, the permanent current constituent may be a major factor in determining the distribution of pollutant dispersion, especially in the case of pollutants that do not decompose quickly in seawater and have a long residence time.

That is, tidal currents in coastal areas may be generally periodic reversing currents whose direction changes according to the tidal cycle, but the averaged flow velocity during the tidal cycle generally may not become zero, and the averaged flow velocity during the tidal cycle may be called tidal residual currents, which may be calculated from the results of tidal current modeling.

Also, in the case of properly reflecting a permanent current involved in ocean currents in the hydrodynamic model in addition to tidal currents, the mean flow by long-term mooring actual measurements should be used in the model in principle, but it may be practically difficult to make an actual observation for permanent currents in various places and for this there may be a method of adjusting the average water level of the sea level boundary as a way to reproduce permanent currents on the model.

More specifically, when there are two or more open sea boundary areas within a model area and the average level of each open sea boundary is different, the values of permanent currents flowing from high water level to low water level may be calculated at all grids.

Moreover, a method of adjusting the average water level of each grid at one open sea boundary area may be applied, and in any case, the model may need to be adjusted so that the size of the reproduced permanent currents matches the actually measured permanent currents within the model area.

Continuously explaining the validation of the hydrodynamic model, the validation by contrast with observed values may be necessary in order to confirm the validity of the hydrodynamic modeling, and there may be several methods of verifying the hydrodynamic modeling for this purpose.

More specifically, first, there may be a sea level displacement comparison method, that is, the flow of the semi-enclosed bay may be calculated using the sea level displacement of the open sea as an input in the hydrodynamic modeling, and at this time not only the flow over time but also the sea level displacement may be calculated in the hydrodynamic model such that the validity of the model may be verified by comparing the actual sea level variability at the observation point of the semi-enclosed bay with the sea level variability calculated by the modeling.

This method may be often utilized for large area modeling, but have a disadvantage of being less useful in narrow-area modeling because the sea level displacement is almost simultaneous at all grid points in modeling narrow coastal waters.

Next, there may be a validation method using Euler's current observation that verifies the validity of the model by comparing the values of the continuous current observation by a current velocity meter moored at a specific point in a model area with the values of the seawater flow simulated by the model.

This method may be frequently used to verify the validation of modeling in coastal areas, and sometimes a method of comparing the degree of the tidal current ellipse by the actually measured flow velocity with the degree of the tidal current ellipse by the modeling may be used.

Subsequently, there may be a validation method using Lagrangian's current observation which compares the data of tracing the drifting buoys with the Lagrangian trajectory of the modeled flow.

In the past, Lagrangian validation methods were not widely used due to the practical difficulty of tracing the movement of drifting buoys for a long period of time by the sextant positioning that is limited by the distance and visibility from the coast, but in recent years, the use of Lagrangian validation methods may be increasing because the Lagrangian's flow observation is possible for a long period of time by using the Global Positioning System (GPS) or Decca Trisponder, which is an advanced navigation device, and particularly the Lagrangian validation method may be more effective in the validation of hydrodynamic models related to pollutant dispersion because the tracing of drifting buoys is not only used to verify the validation of the hydrodynamic model but also is used to estimate pollutant dispersion.

Continuously explaining finite difference diffusion modeling, first, the diffusion equation of pollutants in the Euler coordinate system may be expressed as [Equation 3] below.

$$\frac{\partial C}{\partial t} + U \frac{\partial C}{\partial x} + V \frac{\partial C}{\partial y} + W \frac{\partial C}{\partial z} = \frac{\partial }{\partial x} [ K x \frac{\partial C}{\partial x} ] + \frac{\partial }{\partial y} [ K y \frac{\partial C}{\partial y} ] + \frac{\partial }{\partial z} [ K z \frac{\partial C}{\partial z} ] + Q \qquad [ Equation 3 ]$$

where C represents the concentration of pollutants, t represents time, U, V, and W respectively represent the flow velocity of the mean flow (environmental flow) in the x, y, and z directions, and Kx, Ky, and Kz respectively represent the eddy diffusivity coefficient in the x, y, and z directions, and Q represents an influx rate (source/sink) of pollutants.

Moreover, the terms on the left side of the diffusion equation shown in [Equation 3] above may represent local and advective changes of pollutants, and the right terms may represent the changes due to the turbulent dispersion and the source and sink.

Meanwhile, the diffusion equation of pollutants from the Lagrange perspective following the flow of fluid may be given by [Equation 4] below.

$$\frac{d C}{d t} = \frac{\partial }{\partial x} [ K x \frac{\partial C}{\partial x} ] + \frac{\partial }{\partial y} [ K y \frac{\partial C}{\partial y} ] + \frac{\partial }{\partial z} [ K z \frac{\partial C}{\partial z} ] + Q \qquad [ Equation 4 ]$$

where dC/dt is the change rate of pollutants while following the flow, and may be expressed as [Equation 5] below.

$$\frac{d C}{d t} = \frac{\partial C}{\partial t} + U \frac{\partial C}{\partial x} + V \frac{\partial C}{\partial y} + W \frac{\partial C}{\partial z} \qquad [ Equation 5 ]$$

As mentioned above, there may be two types of diffusion modeling: modeling in the Eulerian coordinate system and modeling in the Lagrangian coordinate system, and the finite difference method may be often used in diffusion modeling, but the finite difference method may have the following problems.

First, as a numerical diffusion problem of advection terms, there appears a spatial oscillation of the concentration distribution and wiggles where the distribution pattern is uneven and bumpy when the advection term of the diffusion equation is differentiated and modeled using the central divided difference method.

To solve this spatial wiggle problem, the upstream difference (or upwind difference) method may be often used in engineering applications, but the problem of numerical diffusion caused by differentiating advection terms may be “inherently” involved in addition to physical dispersion when the upstream difference is used.

In order to solve the numerical diffusion problem of advection terms as described above, a flux-corrected transport (FTC) model that artificially inputs anti-diffusion coefficients negatively in the process of numerical diffusion calculations has been proposed, but the method may be rarely utilized due to the complexity of the algorithm.

Next, there may be a problem of specifying pollution intensity, and generally, concentration values in diffusion finite difference modeling may be calculated only on grids, and the concentration between a grid and a grid may be treated as a linear value with respect to values on adjacent grids.

In addition, in order to properly specify the concentration of the pollutant in the finite difference model on the corresponding grid, the size of the grid may be required to be less than or equal to at least ¼ of the size of a pollution source, that is, for example, when the size of the pollutant outlet is 1 m², the grid of the finite difference should be less than or equal to 20 cm in order to properly specify the concentration of a pollution source, but there may be a problem that it is “practically impossible” to construct such a fine grid in ocean diffusion modeling.

Moreover, when explaining how to specify the intensity of a pollution source, there may be various ways to properly process the intensity of the pollution source in the diffusion model to match reality, as follows.

More specifically, first, there may be a method of using a partial fine grid, which uses a fine grid near a pollution source and a relatively large grid in areas far from the pollution source to represent the intensity of the pollution source in the model in a way to be similar to reality, but this method may have a problem in that it is not simple to connect two grids in finite difference modeling that simultaneously uses grids of different sizes, causing undesirable problems in the “connection part”.

Next, there may be a method of combining the Puff model and the finite difference model, where the puff model is the diffusion model that regards the pollutants continuously discharged from a pollution source as a “lump” of pollutants at regular intervals and figures out the overall pattern of diffusion by combining the analytical solution of the diffusion for each lump, such that when the spatial size of the pollution source is smaller than the grid spacing, the solution of the diffusion distribution is obtained by the Gauss diffusion law using the Puff concept near the pollution source, and then the analytical results may be used as input conditions for the finite difference grid after the size of the puff becomes the size that spans more than one grid in the model grid.

Additionally, there may be a tracer method, and conventionally, dye diffusion experiments may be widely used as physical diffusion experiments which utilize dyes as physical tracers.

That is, for example, the pattern of diffusion may be shown by discharging tens of thousands of pieces of cork instead of dye and observing how those corks disperse, and in a similar way, the dispersion may be reconstructed through a computer by discharging a numerical tracer instead of a physical tracer at the pollution source and then by tracing the location of each particle to calculate the diffusion, which is called a numerical tracer technique.

In the numerical tracer technique, the advective movement of particles by the mean flow such as tidal currents or ocean currents may use the results of the hydrodynamic model, but the eddy dispersion involved in turbulent flows may use random numbers for modeling, so the numerical tracer technique may be usually referred to as the Monte Carlo method.

That is, the present disclosure may simulate the marine dispersion of suspended sediments (SS) and COD using the Q-DISP model that is a diffusion model using the Monte Carlo method, where the Q-DISP model is a diffusion modeling program developed and coded by the inventors of the present disclosure and is named as a meaning of quick dispersion modeling because the results of modeling may be output faster than other conventional diffusion modeling methods.

In the following, the operation principle and processing procedure of the Q-DISP model according to the present disclosure will be described.

First, explaining the advection and dispersion of pollutants, the Q-DISP model may be configured to discharge numerical particles corresponding to the inflow and movement of pollutants, trace the location of each particle moving by tidal currents, wind-driven currents, and turbulent currents, perform a simulation for sufficient actual time to reach an equilibrium state and then calculate the concentration of pollutants from the number of particles scattered in a unit volume.

More specifically, when the new position of the particles in the location (xo, yo) at time t is (xo+δx, yo+δy) after moving for δt hours by the mean flow (tidal currents and permanent currents) and turbulent eddies, the displacement (δx, δy) during the time δt may be expressed as [Equation 6] below.

$$δ x = ( U + {u}^{′} ) δ t \qquad [ Equation 6 ]$$

$$δ y = ( V + {v}^{′} ) δ t$$

where (U, V) is the mean flow velocity calculated by the hydrodynamic model, and (u′, v′) is the turbulent flow velocity due to eddies.

In addition, since the turbulent flow velocity constituents u′ and v′ are irregularly distributed within the range of U_{b} and V_{b}, the turbulent characteristic flow velocity which is determined by the strength of the turbulent field, the displacement during the time δt may be expressed as [Equation 7] below when modeling the turbulent flow velocity using the Monte Carlo method.

$$δ x = ( U + μ {U}_{b} ) δ t \qquad [ Equation 7 ]$$

$$δ y = ( V + {vV}_{b} ) δ t$$

where μ and v are respectively random numbers uniformly distributed in the range of −1 and 1.

Meanwhile, the sizes of the turbulent characteristic flow velocities U_{b} and V_{b} may be respectively related to the eddy diffusion coefficients Dx and Dy in the x and y directions as shown in [Equation 8] below, so the displacement during the time δt according to turbulent flow may be known by knowing the diffusion coefficient, and the eddy diffusion coefficient may be calculated by knowing the turbulent displacement.

$${U}_{b} = {( 6 Dx / δ t )}^{1 / 2} \qquad [ Equation 8 ]$$

$${V}_{b} = {( 6 Dy / δ t )}^{1 / 2}$$

Continuingly explaining the estimation of the eddy diffusion coefficient, the size of the eddy diffusion coefficient involved in turbulent flow may be not constant and the eddy diffusion coefficient may increase as the spatial and temporal size of the turbulent field increases, such that an eddy diffusion coefficient may be estimated from dispersion experiments of buoys.

That is, the neighbor diffusion coefficient D(σ) involved in the variance σ² of the distance between buoys during time t may be expressed as [Equation 9].

$$D ( σ ) = {σ}^{2} / 2 t \qquad [ Equation 9 ]$$

Next, when explaining the modeling of suspended sediments (SS) and COD decomposition, suspended sediments (SS) flowing into the ocean may gravitate to the bottom over time, and COD gradually may decompose over time, so that the modeling techniques according to the sedimentation of SS and the decomposition of COD may be as follows.

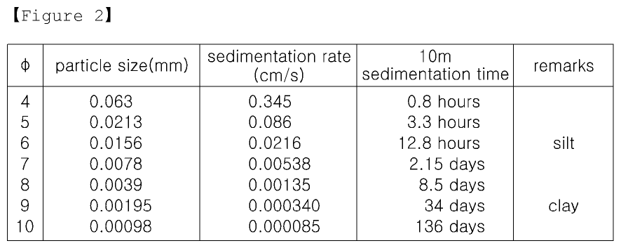

That is, referring to FIG. 2, FIG. 2 is a table showing the sedimentation rate according to the particle size of suspended sediments having a specific gravity of 2.65 in seawater with a water temperature of 20° C.

More specifically, the sedimentation rate of suspended sediments, which is determined by the particle size and specific gravity, may be generally faster as the particle size and the specific gravity increase and as shown in the table in FIG. 2, it may take 12.8 hours for slit particles with the sedimentation rate of 0.0216 cm/sec to sink 10 meters and in the case of clay with much smaller particle sizes take 34 to 136 days to sink 10 meters in laboratory conditions, but in the real ocean, it may be estimated that even clay will sink within a few weeks due to the flocculation of suspended sediments caused by biological and chemical actions.

That is, in the real ocean where the effects of tidal currents, turbulent currents, sea waves and the like act in combination, the sedimentation rate of suspended sediments may be affected not only by the size and the specific gravity of particles, but also by various factors such as the degree of turbulence, the average flow velocity in the vertical direction, and the shape of a particle.

More specifically, the number of suspended sediment particles N per water column of a unit area by the sedimentation of suspended sediments in seawater may be expressed as the following [Equation 10], and the attenuation coefficient α may increase as the sedimentation rate increases.

$$\frac{d N}{d t} = - α N \qquad [ Equation 10 ]$$

In addition, according to [Equation 10] above, the number of suspended sediment particles N(t) at time t may be expressed as [Equation 11] below.

$$N ( t ) = {N}_{0} exp ( - α t ) = {N}_{0} exp ( \frac{- t}{{t}_{0}} ) \qquad [ Equation 11 ]$$

where N₀ is the initial number of particles, and t₀(t₀=1/α) is an exponential half-life (e-folding time) or residence time taken for the number of particles to decrease by e⁻¹ (=0.368) times from the original value.

Meanwhile, when the exponential attenuation equation dN/dt=−αt for the number of particles in seawater is finitely differentiated, the following relationship shown in [Equation 12] below may be established between the number of particles N(n+1) at time (t+δt) and the number of particles N(n) at the time t.

$$N ( n + 1 ) = N ( n ) ( 1 - α δ t ) \qquad [ Equation 12 ]$$

Therefore, whether to be accumulated by the sedimentation of suspended sediment particles may be modeled by treating the particles as being accumulated when the uniformly distributed random number arbitrarily taken between 0 and 1 is within the range of 0 and αδt and by treating the particles as floating when the uniformly distributed random number is within the range of αδt and 1, and additionally the concentration reduction by the decomposition of COD in seawater may be modeled using uniform random numbers in a similar way.

Next, explaining the concentration calculation of pollutants, the concentration of pollutants in seawater may be calculated from the number of numerical tracers in each grid, that is, for example, when the discharge of Q kg of pollutants per unit time is modeled as the discharge of m particles per unit time in the diffusion model, one particle may represent the amount of pollutants in Q/m kg.

In addition, when n particles are placed in a water column where the width, length, and depth are respectively δx, δy, and δz, the mass of pollutants in the water column may be nQ/m kg, so the concentration C may be expressed as [Equation 13] below.

$$C = \frac{n Q}{m δ x δ y δ z} \qquad [ Equation 13 ]$$

Therefore, the SS concentration or COD concentration in seawater may be quantitatively calculated using this relationship.

Continuously explaining an equilibrium state of dispersion, when the discharge of a pollution source continues persistently, the concentration of pollutants in seawater may initially increase over time, but after sufficient time has elapsed, the amount of incoming load from the pollution source may reach the same state with the amount of pollutants escaping from the seawater by the decomposition or sedimentation, so that an equilibrium state is reached in which the dispersion concentration in seawater no longer increases even when there is a continuous pollution load.

In addition, the time to reach the equilibrium state may vary depending on the exponential half-life or residence time by the decomposition or sedimentation of the pollutants, and generally, more than 95% of the equilibrium state may be reached after 3 or 4 times the residence time of the pollutants have elapsed, so considering this, it may be necessary in diffusion modeling to simulate a sufficient actual time to reach the equilibrium state.

That is, the diffusion modeling of suspended sediments with a sedimentation time of about one week may require more than one month of simulation, the diffusion modeling of COD that takes about one month to decompose may require to model more than 100 days of actual time, and in the case of thermal discharge modeling, the degree of heat exchange through the sea surface should be considered and generally, the equilibrium state may be reached only after modeling for an actual period of 100 days or more.

Moreover, the time to reach the equilibrium state in pollutant dispersion modeling may vary depending on the target sea area of the model, that is, when only the area very close to the pollutant outlet is set as the model area, the equilibrium state will be reached after a relatively short period of time by the balance between the amount of the pollutant load flowing into this area and the amount of the pollutants escaping from this area through the outer boundary, but the wider the target sea area of the model is, the more time it takes for the model area to reach the equilibrium state as a whole, such that the equilibrium state is reached only after several times the exponential half-time of the pollutant decomposition (sedimentation) have elapsed when the model area is sufficiently large.

Next, explaining the utilization of periodicity in tidal waters, when numerical tracers are continuously discharged with the continuously discharged pollutant load, the number of particles to be traced may increase over time, so modeling the pollutant dispersion behavior over several months may require extensive computer memory and computation time, thereby making it difficult to process even on large computers.

On the other hand, when the flow field is steady currents or periodically reversing currents, this problem may be overcome, and the principle is as follows.

More specifically, in the case of steady currents as in rivers, the “equivalent” effect of tracing continuously discharged particles may be obtained by “remembering” the path of the initially discharged particles without discharging continuously numerical tracers from the pollution source in simulating the dispersion behavior according to the continuous pollution discharge.

That is, for example, when the locations of 100 particles discharged all at once are remembered after 8, 9, and 10 seconds, this information may be roughly equivalent to the diffusion states after 7, 8, and 9 seconds for particles discharged 1 second later, and also be roughly equivalent to the diffusion states after 6, 7, and 8 seconds for particles discharged 2 seconds later.

Therefore, using this principle of equivalence, Monte Carlo diffusion modeling may be possible by tracing only the particles discharged at once in the case of continuous pollution discharge in a steady flow.

More specifically, when considering only permanent currents and periodic tides in coastal areas, computer memory capacity and performance time may be drastically reduced by continuously tracing each particle at each δt time taking into account only the particles discharged during one tidal cycle using the periodicity of the tides and simultaneously by using the method of remembering the location of a particle according to time.

That is, for example, the location of the particles after 0.2 tidal cycle after discharge may be treated as statistically equivalent to the location where the particles discharged before 1.2 tidal cycle have already passed before 1 tidal cycle, and this logic of “periodic repetition” may be applied when the number of discharged particles is sufficiently large although each particle is not repeatedly placed exactly at the same location every tidal cycle.

In addition, since real tides have phenomena of spring tides and neap tides, the above principle of periodic repetition may be not strictly applied, and applying the principle of periodic repetition may assume that the tidal currents of the ocean tide are periodically repeating the flow state of the middle tide between spring tides and neap tides ignoring phenomena of spring tides and neap tides.

Continuously explaining the problem of pollutant inflow and outflow outside the model area, information on seawater flow may be essential in the pollutant dispersion modeling, so it may be necessary to consider a sufficiently large area as a model area considering the dispersion distribution of pollutants in the equilibrium state.

Moreover, when simulating for a long period of actual time, there may be particles that fall outside the model area, and the particles that fall outside the model area may have the possibility of being reintroduced into the model area rather than being dissipated particles.

In consideration of this, it may be necessary to set a buffer outside the hydrodynamic model area in the pollutant dispersion model, such a buffer may have a sufficient spatial size, and the flow in the buffer may be set in advance taking into account physical conditions, generally specifying the flow value or extrapolation value adjacent to the outside of the model area.

Next, explaining the case where various pollutants having different residence times are mixed, suspended sediments (SS), which are suspended soils generated during dredging or landfill construction, may be mixed with particles of various sizes, and the residence time of all particles may not be considered the same since the sedimentation rate of suspended sediments varies depending on the size of the particles.

Even in this case, dispersion may have to be simultaneously modeled in Monte Carlo diffusion modeling by setting various residence times corresponding to the composition ratio of each particle size and for this, each particle's “life” may be set in advance when setting up particles at the pollution source, and the question of whether each particle dissipates or persists in seawater may be determined by a comparison method with the uniform random number described above.

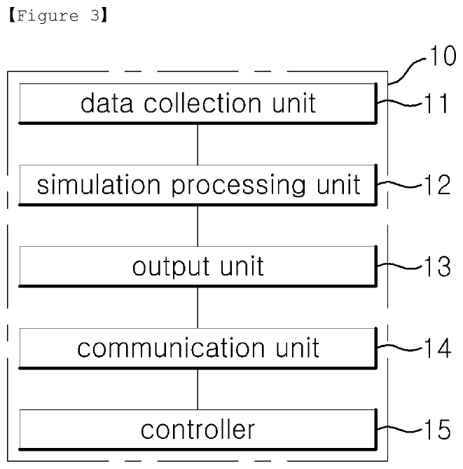

Therefore, it may be possible to implement the system and method for simulations of marine pollution dispersion using a numerical tracer technique according to an exemplary embodiment of the present disclosure from the above-described contents, that is, referring to FIG. 3, FIG. 3 is a block diagram schematically showing the overall configuration of the marine pollution dispersion simulation system 10 using the numerical tracer technique according to an exemplary embodiment of the present disclosure.

As shown in FIG. 3, the marine pollution dispersion simulation system 10 using the numerical tracer technique according to the exemplary embodiment of the present disclosure may be broadly configured to include a data collection unit 11 that performs a process of collecting various predetermined data to perform simulations on the marine pollution dispersion, a simulation processing unit 12 that performs a process of performing simulations on the marine pollution dispersion according to predetermined settings using the numerical tracer technique on the basis of data collected through the data collection unit 11, and an output unit 13 that performs a process of outputting various data, each processing procedure and processing results collected and processed through the data collection unit 11 and the simulation processing unit 12 according to predetermined settings.

As shown in FIG. 1, the system 10 may further include a communication unit 14 that performs a process of transmitting and receiving various data by communicating with external devices such as servers or user terminals through at least one of wired or wireless communication, and a controller 15 that performs a process of controlling the overall operations of each unit and the system 10. The controller 15 may include a processor and a memory storing instructions to be executed by the processor. Each of the data collection unit 11, the simulation processing unit 12, and the output unit 13 may be program modules to be executed by the controller 15. The communication unit 14 may be any communication hardware device for sending and/or receiving any wired or wireless communication.

More specifically, first, the data collection unit 11 may be configured to receive basic data, such as, for example, grids, water depths, tides, and tidal currents, for implementing the hydrodynamic model of the area to be simulated, the data on environmental conditions or the data on a pre-implemented hydrodynamic model for the corresponding area through a separate input means directly or externally from the outside through wired or wireless communication.

In addition, the simulation processing unit 12 may perform a process of performing simulations on the marine pollution dispersion using the numerical tracer technique by calculating the concentration of pollutants from the number of particles scattered within a unit volume after performing the simulation for a sufficient actual time to reach the equilibrium state while discharging numerical particles corresponding to inflow and movement and tracing the location of each particle moving by tidal currents, wind-driven currents, and turbulent currents by referring to from [Equation 1] to [Equation 13] on the basis of the data received through the data collection unit 11.

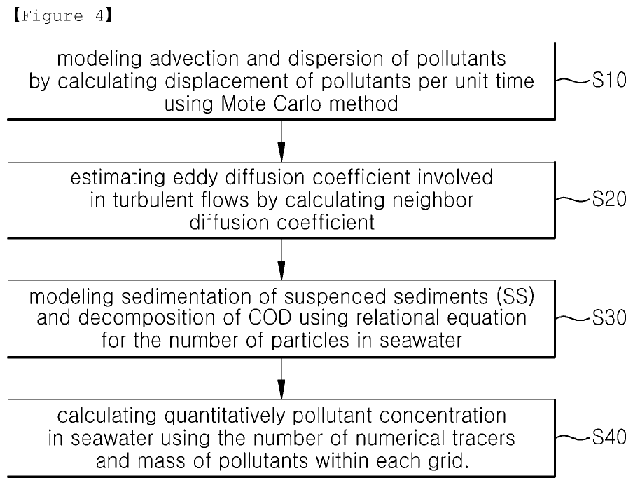

More specifically, referring to FIG. 4, FIG. 4 is a flowchart schematically showing the overall processing procedure performed in the simulation processing unit 12 of the marine pollution dispersion simulation system 10 using the numerical tracer technique according to an exemplary embodiment of the present disclosure shown in FIG. 3.

As shown in FIG. 4, the processing procedure of the marine pollution dispersion simulation using the numerical tracer technique performed in the simulation processing unit 12 may largely configured to include a step of pollutant dispersion modeling (S10) in which the advection and dispersion of pollutants is modeled by calculating the displacement of pollutants for a unit time using Monte Carlo method, a step of eddy diffusion coefficient calculation (S20) in which the eddy diffusion coefficient involved in turbulent flows is estimated by calculating the neighbor diffusion coefficient, a step of pollutant sedimentation and decomposition modeling (S30) in which the sedimentation of suspended sediments (SS) and the decomposition of COD is modeled using a relational equation for the number of particles in seawater, and a step of pollutant concentration calculation (S40) in which the pollutant concentration in seawater is quantitatively calculated using the number of numerical tracers and the mass of pollutants within each grid.

As described above with reference from [Equation 6] to [Equation 8], the step of pollutant dispersion modeling (S10) described above may be configured to calculate the displacement (δx, δy) during the time δt by modeling the turbulent flow velocity using the Monte Carlo method when the new location of the particles at the location (xo, yo) at time t is (xo+δx, yo+δy) after the time δt when moving by the mean flow (turbulent currents and permanent currents).

Moreover, the step of eddy diffusion coefficient calculation (S20) may be configured to calculate the neighbor diffusion coefficient D(σ) according to the variance σ² of the distance between buoys during the time t, as described above with reference to [Equation 9].

Furthermore, as described above with reference to FIG. 2 and the equations from [Equation 10] to [Equation 12], the step of pollutant sedimentation and decomposition modeling (S30) may be configured to calculate the number (N) of suspended sediment particles per water column of a unit area and the number N(t) of suspended sediment particles at the time t by the sedimentation of suspended sediments present in the seawater, and to model both whether to be accumulated by the sedimentation of the suspended sediment particles and the concentration reduction by the decomposition of COD in seawater using the relational equation between the number of particles N(n+1) in the time (t+δt) and the number of particles N(n) in the time t on the basis of the values of uniformly distributed random numbers.

That is, the step of pollutant sedimentation and decomposition modeling (S30) may model the behavior of suspended sediment particles by treating the particles as being accumulated when the uniformly distributed random number taken arbitrarily between 0 and 1 is within the range of 0 and αδt and by treating the particles as continuously floating when the uniformly distributed random number is within the range of αδt and 1.

In addition, as described above with reference to [Equation 13], the step of pollutant concentration calculation (S40) may be configured to model the discharge of Q kg of pollutants per unit time as the discharge of m particles per unit time, wherein one particle represents the amount of pollutants in Q/m kg, and to calculate the concentration (C) of pollutants in seawater from the number of numerical tracers present in each grid on the basis of the fact that the amount of pollutants present in the water column is nQ/m kg when there are n particles in the water column where the width, length, and depth are respectively δx, δy, and δz.

Moreover, the simulation processing unit 12 may be configured to perform simulations for sufficient actual time according to predetermined settings in consideration of the time to reach the equilibrium state in which the dispersion concentration of pollutants in the seawater no longer increases, and to model the pollutant dispersion by equivalently estimating the dispersion state of continuously discharged particles over time on the basis of a time-specific diffusion state of the initially discharged particles without discharging continuously numerical tracers from the pollution source in a diffusion simulation according to the continuous pollutant discharge when the flow field is steady currents or periodic reversing currents, and accordingly, the work load of the computation and execution time required for modeling may be reduced by tracing only the particles discharged during one tidal cycle and then by equivalently modeling the subsequent tidal cycles using the periodicity of the tide.

Moreover, the simulation processing unit 12 may be configured to model the pollutant dispersion by setting a buffer outside the hydrodynamic model area according to predetermined settings in order to consider the possibility that particles escaping from the predetermined hydrodynamic model area may be reintroduced into the model area again, and to model each pollutant particle simultaneously by setting a different residence time according to a composition ratio of each pollutant particle size and specifying a lifetime of each particle in advance when multiple pollutants having different residence times are mixed.

In addition, the output unit 13 may be configured to perform a process of visually displaying various information including various data received through the data collection unit 11 as described above and the processing procedure and analysis results of the simulation processing unit 12 through a monitor or display.

As described above, the output unit 13 may be configured to visually display various information through a monitor or display, and audibly transmit various information through an audio output means such as a speaker at the same time, and the present disclosure may be not necessarily limited to the configurations presented in the exemplary embodiments of the present disclosure, but may be configured in various ways as necessary by those skilled in the art without departing from the spirit and essence of the present disclosure.

Moreover, the system 10 may be configured to transmit various data collected through the data collection unit 11 and various information including the processing procedure and the results of the simulation processing unit 12 through the communication unit 14 according to the control of the controller to an external device such as a server or a user terminal, thereby easily implementing a simulation and information supply service system and method that provides a marine pollution dispersion simulation and a variety of related information in a customized manner according to the user's request.

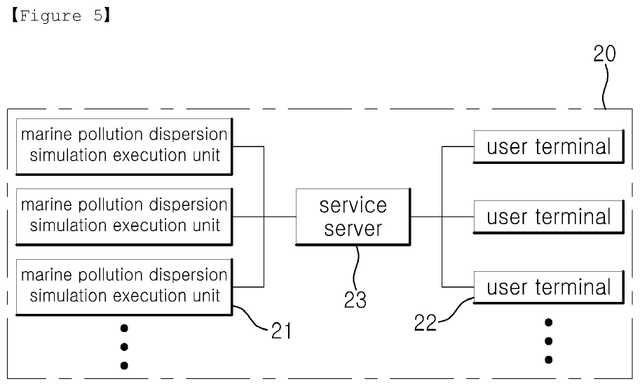

That is, referring to FIG. 5, FIG. 5 is a block diagram schematically showing the overall configuration of the marine pollution dispersion simulation service supply system 20 configured to provide customized marine pollution dispersion simulation and various information related thereto using the system 10 and method for simulation of the marine pollution dispersion using the numerical tracer technique according to an exemplary embodiment of the present disclosure configured as described above.

As shown in FIG. 5, the marine pollution dispersion simulation service supply system 20 according to an exemplary embodiment of the present disclosure may be configured to include a marine pollution dispersion simulation execution unit 21 that performs a simulation after receiving various information for the marine pollution dispersion simulation and builds a database related to the marine pollution dispersion simulation for each area by collecting various data including the received information for the simulation and the simulation results, a user terminal 22 for a user to input various information for a marine pollution dispersion simulation and to request and receive desired services, and a service server 23 that receives the simulation results of the marine pollution dispersion and various data related thereto from each of the simulation execution unit 21 and provides the corresponding service according to the information received from the user terminal 22 and the user's request while being interlocked with the marine pollution dispersion simulation execution unit 21 and the user terminal 22.

As described above with reference to FIGS. 3 and 4, the simulation execution unit 21 may be configured to use the marine pollution dispersion simulation system 10 using the numerical tracer technique according to an exemplary embodiment of the present disclosure.

Moreover, the user terminal 22 may be configured to include, for example, a personal portable information communication terminal such as a smartphone or tablet PC, or an information processing device such as a PC or laptop.

Therefore, as described above, it may be possible to implement the system and method for simulations of marine pollution dispersion using the numerical tracer technique according to an exemplary embodiment of the present disclosure, and accordingly the Q-DISP (Quick Dispersion) model, which is a diffusion model using the Monte Carlo method may be possible to model faster than conventional diffusion modeling methods and to perform simulations through the numerical tracer technique with respect to a long-term continuous discharge of marine pollution dispersion according to the present disclosure, thereby solving the problems of conventional systems and methods for simulations of marine pollution dispersion using the numerical tracer technique, which have limitations that make it difficult to be applied to pollutants that are continuously discharged for a long period of time because a high-performance computer and a large amount of memory are required.

As described above, the details of the system and method for simulations of marine pollution dispersion using the numerical tracer technique according to the present disclosure have been described through the exemplary embodiments of the present disclosure, but the present disclosure may be not limited to the contents described in the above exemplary embodiments, and therefore, it is natural that the present disclosure may be modified, changed, combined, and replaced according to various design needs and various other factors by those of ordinary skill in the technical field to which the present disclosure belongs.

What is claimed is:

1. A system for a simulation of a marine pollution dispersion using a numerical tracer technique, the system comprising:
a data collection unit that performs a process of collecting various data predetermined in order to perform the simulation on the marine pollution dispersion;
a simulation processing unit that performs the simulation on the marine pollution dispersion according to a predetermined setting using the numerical tracer technique on a basis of various data collected through the data collection unit; and
an output unit that performs a process of outputting various data, each processing procedure, and a processing result that are collected and processed through the data collection unit and the simulation processing unit according to the predetermined setting.

2. The system of claim 1, further comprising:
a communication unit that performs a process of communicating with an external device including a server or a user terminal through at least one of a wired and wireless communication in order to transmit and receive various data; and
a controller that performs a process of controlling an overall operation of the system.

3. The system of claim 1, wherein the data collection unit performs a process of receiving through a separate input means directly or externally from an outside a basic data such as grids, depths, tides and current information for implementing a hydrodynamic model of a region to be simulated, a data on environmental conditions, or a data on a pre-implemented hydrodynamic model about the corresponding region.

4. The system of claim 1, wherein the simulation processing unit performs a process comprising:
a step of pollutant dispersion modeling in which an advection and dispersion of pollutants are modeled by calculating a displacement of pollutants per unit time using a Monte Carlo method,
a step of eddy diffusion coefficient calculation in which an eddy diffusion coefficient involved in turbulent flows is estimated by calculating a neighbor diffusion coefficient,
a step of pollutant sedimentation and decomposition modeling in which a sedimentation of suspended sediments (SS) and a decomposition of COD are modeled using a relational equation for a number of particles in seawater, and
a step of pollutant concentration calculation in which a pollutant concentration in seawater is quantitatively calculated using a number of numerical tracers in each grid and a mass of pollutants.

5. The system of claim 4, wherein using the following mathematical equations, the step of pollutant dispersion modeling performs a process of calculating the displacement (δx, δy) during a time δt by modeling turbulent flow velocity using the Monte Carlo method when particles at a location (xo, yo) at the time t move by a mean flow (tidal currents and permanent currents) and turbulent vortices, and then locate at a new position (xo+δx, yo+δy) after δt hours, and
wherein
$$δ x = ( U + μ {U}_{b} ) δ t$$
$$δ y = ( V + {vV}_{b} ) δ t$$
$${U}_{b} = {( 6 Dx / δ t )}^{1 / 2}$$
$${V}_{b} = {( 6 Dy / 6 t )}^{1 / 2}$$
where U and V are the velocity of the mean flow calculated by the hydrodynamic model, μ and v are respectively random numbers uniformly distributed in a range of −1 and 1, U_{b} and V_{b} are turbulent characteristic velocities, and Dx and Dy are respectively eddy diffusion coefficients in the x and y directions.

6. The system of claim 4, wherein the step of eddy diffusion coefficient calculation performs a process of calculating a neighbor diffusion coefficient D(σ) according to a variance σ² of a distance between buoys during the time t using the following mathematical equation.
$$D ( σ ) = {σ}^{2} / 2 t$$

7. The system of claim 4, wherein the step of pollutant sedimentation and decomposition modeling performs a process of:
calculating a number of suspended sediment particles (N) per water column of a unit area due to the sedimentation of suspended sediments present in the seawater and the number of suspended sediment particles N(t) at a time t, using the following mathematical equation,
$$\frac{d N}{d t} = - α N$$
$$N ( t ) = {N}_{0} exp ( - α t ) = {N}_{0} exp ( \frac{- t}{{t}_{0}} )$$
where α is a attenuation coefficient, N₀ is an initial number of particles, and t₀(t₀=1/α) means an exponential half-life (e-folding time) or residence time, respectively, for the number of particles to be reduced by a factor of e⁻¹(=0.368), and
modeling whether to be accumulated by the sedimentation of suspended sediment particles and the concentration reduction according to the decomposition of COD in seawater on the basis of a value of a uniformly distributed random number using a relationship between the number of particles N(n+1) in the time (t+δt) and the number of particles N(n) in the time t, which is expressed by the following equation.
$$N ( n + 1 ) = N ( n ) ( 1 - α δ t )$$

8. The system of claim 7, wherein the step of pollutant sedimentation and decomposition modeling performs a process of modeling a behavior of suspended sediment particles
by treating the corresponding particles as being accumulated when a uniformly distributed random number taken arbitrarily between 0 and 1 is within an interval between 0 and αδt, and
by treating the particles as continuously floating when the uniformly distributed random number is within the interval between αδt and 1.

9. The system of claim 4, wherein the step of pollutant concentration calculation performs a process of:
modeling a discharge of Q kg of pollutants per unit time as a discharge of m particles per unit time, wherein one particle represents an amount of pollutants in Q/m kg, and
calculating the concentration (C) of pollutants in the seawater from a number of numerical tracers present in each grid using the following mathematical equation on the basis of a fact that the mass of pollutants present in a water column is nQ/m kg when there are n particles in the water column where width, length, and depth of the water column are respectively δx, δy, and δz.
$$c = \frac{n Q}{m δ x δ y δ z}$$

10. The system of claim 4, wherein the simulation processing unit performs the simulation for actual time according to predetermined settings, taking into consideration a time to reach an equilibrium state in which a dispersion concentration of pollutants in seawater no longer increases.

11. The system of claim 4, wherein the simulation processing unit performs a process of modeling the pollutant dispersion
not by continuously discharging numerical tracers from a pollution source
but by estimating equivalently a diffusion state of later continuously discharged particles over time on the basis of a time-specific diffusion state of the initially discharged particles
while simulating the diffusion according to continuous discharges of pollutants when a flow field is steady currents or periodically reversing currents,
thereby reducing an amount of computational work and execution time required for modeling by tracing only the particles discharged during one tidal cycle using a periodicity of the tide and then by equivalently modeling a subsequent tidal cycle.

12. The system of claim 4, wherein the simulation processing unit performs a process of modeling the pollutant dispersion by setting a buffer outside a hydrodynamic model area according to predetermined settings in order to consider a possibility that particles escaping from the hydrodynamic model area reenter the corresponding model area again.

13. The system of claim 4, wherein the simulation processing unit performs a process of simultaneously modeling each pollutant particle by
setting a different residence time for each pollutant particle according to a composition ratio per size of each pollutant particle and
specifying a lifetime of each particle in advance
when multiple pollutants having different residence times are mixed.

14. The system of claim 1, wherein the output unit performs a process of visually displaying various data received through the data collection unit and various information including the processing procedure and the result of the simulation processing unit through a monitor or display.

15. The system of claim 14, wherein the output unit performs a process of:
visually displaying various information through a monitor or display, and at the same time
audibly transmitting various information through an audio output means including a speaker.

16. The system of claim 2, wherein the controller performs a process of transmitting various data received through the data collection unit and various information including the processing procedure and analysis results of the simulation processing unit to an external device including a server or a user terminal according to predetermined settings through the communication unit.

17. A method for a simulation of a marine pollution dispersion using a numerical tracer technique, the method comprising:
a step of system construction that performs a process of constructing a simulation system where the simulation of the marine pollution dispersion is performed using the numerical tracer technique; and
a step of simulation execution that performs the simulation of the marine pollution dispersion using the simulation system,
wherein the simulation system uses the system for the simulation of the marine pollution dispersion using the numerical tracer technique according to claim 1.

18. A marine pollution dispersion simulation service supply system, the system comprising:
a marine pollution dispersion simulation execution unit that performs a process of performing simulations after receiving various information for simulations of marine pollution dispersion, and building a database related to simulations of marine pollution dispersion for each region by collecting various data including information received for simulations and a simulation result;
a user terminal for each user to input information related to simulations of marine pollution dispersion and request and receive a desired service; and
a service server that performs a process of receiving various data including the simulation results from the simulation execution unit, and providing the corresponding services according to information received from each of the user terminal and the user's request, while being interlocked with the simulation execution unit and the user terminal,
wherein the marine pollution dispersion simulation execution unit uses the system for the simulation of the marine pollution dispersion using the numerical tracer technique according to claim 1.

19. The system of claim 18, wherein the user terminal comprises at least one of a personal portable information communication terminal including a smart phone or a tablet PC, and an information processing device including a PC or a laptop.
